(19) United States
(12) Patent Application Publication
(10) Pub. No.: US 2025/0186774 A1
(43) Pub. Date: Jun. 12, 2025

(54) SYSTEM AND METHOD OF NERVE STIMULATION UTILIZING THORACIC IMPEDANCE

(71) Applicant: The Alfred E. Mann Foundation for Scientific Research, Valencia, CA (US)

(72) Inventors: Brian Mech, Buffalo, MN (US); Neil Talbot, La Crescenta, CA (US)

(21) Appl. No.: 18/968,145

(22) Filed: Dec. 4, 2024

Related U.S. Application Data
(60) Provisional application No. 63/607,444, filed on Dec. 7, 2023.

(51) Int. Cl. A61N 1/36; A61N 1/02; A61N 1/372

(52) U.S. Cl. CPC A61N 1/3611; A61N 1/025; A61N 1/3601; A61N 1/3614; A61N 1/37252

(57) ABSTRACT

A method of treating obstructive sleep apnea in a patient includes measuring, with electrodes, thoracic impedance of the patient, and stimulating, with a nerve stimulation device implanted in the patient, a nerve of patient based on the thoracic impedance. The nerve is associated with sleep apnea, such as the hypoglossal nerve and/or the ansa cervicalis nerve. The electrodes may be either surface electrodes or implanted electrodes, and the thoracic impedance may be trans-thoracic impedance or intra-thoracic impedance.

### CROSS-REFERENCE TO RELATED APPLICATION(S)

The present application claims priority to and the benefit of U.S. Provisional Application No. 63/607,444, filed Dec. 7, 2023, the entire content of which is incorporated herein by reference.

### BACKGROUND

### 1. Field

The present disclosure relates generally to systems and methods of treating obstructive sleep apnea by nerve stimulation.

### 2. Description of the Related Art

Obstructive sleep apnea (OSA) is a sleep-related breathing disorder in which the complete collapse (apnea) or partial collapse (hypopnea) of the upper airway causes a decrease in oxygen saturation and/or arousal from sleep. OSA may result in fragmented, non-restorative sleep and significant impairment of the cardiovascular health and mental health of the individual suffering from OSA.

Some related art systems treat OSA by stimulating the patient's hypoglossal nerve, which causes tongue movement to relieve upper airway obstruction. The delivery of the hypoglossal nerve stimulation is timed with the patient's breathing (i.e., the patient's respiratory cycle). Some related art systems and methods measure the patient's breathing utilizing an implanted pressure sensor or an inertial measurement unit (IMU). However, implanted pressure sensors and IMUs frequently fail to detect every respiratory cycle due to other artifacts in the signal and/or a low signal-to-noise ratio.

The above information disclosed in this Background section is only for enhancement of understanding of the background of the invention and therefore it may contain information that does not constitute prior art.

### SUMMARY

The present disclosure relates to various embodiments of a method of treating obstructive sleep apnea in a patient. In one embodiment, the method includes measuring, with a number of electrodes, thoracic impedance of the patient, and stimulating, with a nerve stimulation device implanted in the patient, a nerve of the patient based on the thoracic impedance. The nerve is associated with sleep apnea.

The nerve stimulated by the nerve stimulation device may be the hypoglossal nerve or the ansa cervicalis nerve of the patient.

Stimulating the nerve may be performed in response to a value of the thoracic impedance indicating the patient started to inhale or is about to inhale.

The method may also include wirelessly transmitting the thoracic impedance from the electrodes to the nerve stimulation device.

The electrodes may be attached to the patient's chest or to the patient's abdomen.

The electrodes may be implanted in the patient.

The electrodes may include a first electrode on a housing of the nerve stimulation device and a second electrode coupled to the nerve stimulation device by a first lead.

The stimulation may be performed with the second electrode.

The stimulation may be performed with a third electrode coupled to the nerve stimulation device by a second lead separate from the first lead.

The stimulation may be performed with a third electrode coupled to the nerve stimulation device by the first lead.

The measuring of the thoracic impedance may include measuring trans-thoracic impedance of the patient or intra-thoracic impedance of the patient.

The method may also include measuring, with an inertial measurement unit of the nerve stimulation device, movement of the patient's chest.

The stimulation may be further based on the movement of the patient's chest.

The method may also include titrating stimulation parameters of the nerve stimulation device based on the motion of the patient's chest.

In another embodiment, a method of treating obstructive sleep apnea in a patient, includes measuring, with at least one bioimpedance electrode, bioimpedance of tissue at a back of the patient's throat, and stimulating, with a nerve stimulation device implanted in the patient, a nerve of the patient based on the bioimpedance. The nerve is associated with sleep apnea.

The nerve stimulated by the nerve stimulation device may be the hypoglossal nerve or the ansa cervicalis nerve of the patient.

Stimulating the hypoglossal nerve may be performed in response to a value of the bioimpedance indicating the patient started to inhale or is about to inhale.

The present disclosure relates to various embodiments of a nerve stimulation device configured to treat obstructive sleep apnea in a patient. In one embodiment, the nerve stimulation device includes a housing, a processor in the housing, a non-volatile memory device in the housing, a controller in the housing, a power supply in the housing, and a number of electrodes coupled to the housing. The non-volatile memory device includes computer-readable instructions which, when executed by the controller, cause the electrodes to measure thoracic impedance of the patient and to deliver electrical stimulation to a nerve of patient based on the thoracic impedance. The nerve is associated with sleep apnea, such as the hypoglossal nerve or the ansa cervicalis nerve of the patient.

The computer-readable instructions, when executed by the controller, may cause the electrodes to deliver the electrical stimulation to the nerve in response to a value of the thoracic impedance indicating the patient started to inhale or is about to inhale.

The computer-readable instructions, when executed by the controller, may cause the controller to wirelessly receive the thoracic impedance from the electrodes.

The electrodes may include a number of surface electrodes configured to be attached to the patient's chest or abdomen.

The electrodes may include a number of implantable electrodes.

The electrodes may include a first electrode on the housing and a second electrode coupled to the housing by a lead.

The electrodes may include a first electrode coupled to the housing by a first lead and a second electrode coupled to the housing by a second lead.

The electrodes may include two electrodes, three electrodes, or more.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable system or method of hypoglossal nerve stimulation.

### BRIEF DESCRIPTION OF THE DRAWINGS

The features and advantages of embodiments of the present disclosure will be better understood by reference to the following detailed description when considered in conjunction with the drawings. The drawings are not necessarily drawn to scale.

FIGS. 1A-1B are schematic views of a nerve stimulation (NS) system including an implantable pulse generator according to one embodiment of the present disclosure;

FIG. 2 is a block diagram of the implantable pulse generator of FIGS. 1A-1B;

FIG. 3 is a schematic view of a system including a nerve stimulation (NS) device including an implantable pulse generator, and a cardiac resynchronization therapy defibrillator (CRT-D) or an implantable cardiovert defibrillator (ICD) in communication with the NS device according to one embodiment of the present disclosure;

FIG. 4 is a block diagram of the implantable pulse generator and the CRT-D or ICD of FIG. 3;

FIG. 5 is a flowchart illustrating tasks of a method of nerve stimulation to treat obstructive sleep apnea according to one embodiment of the present disclosure; and

FIG. 6 is a flowchart illustrating tasks of a method of nerve stimulation to treat obstructive sleep apnea according to another embodiment of the present disclosure.

### DETAILED DESCRIPTION

Aspects of the present disclosure relate to various embodiments of a system and method for performing nerve stimulation (NS) to treat a patient's obstructive sleep apnea utilizing the patient's thoracic impedance, trans-thoracic impedance, or intra-thoracic impedance to control the timing of the NS. The NS may be applied to any nerve associated with sleep apnea, such as the patient's hypoglossal nerve or the patient's ansa cervicalis nerve. Utilizing thoracic impedance as a respiratory signal to time the delivery of nerve stimulation is more reliable and accurate than conventional methods of NS utilizing an inertial measurement unit (IMU) and/or a pressure sensor.

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. As utilized herein, the term “and/or” includes any and all combinations of one or more of the associated listed items.

It will be understood that, although the terms “first”, “second”, “third”, etc., may be utilized herein to describe one or more suitable elements, components, regions, and/or sections, these elements, components, regions, and/or sections should not be limited by these terms. These terms are only utilized to distinguish one element, component, region, layer or section from another element, component, region, layer or section. Thus, a first element, component, region, or section discussed could be termed a second element, component, region, or section, without departing from the spirit and scope of the present disclosure.

It will be understood that when an element is referred to as being “on”, “connected to”, “coupled to”, or “adjacent to” another element, it can be directly on, connected to, coupled to, or adjacent to the other element, or one or more intervening element(s) may be present. In contrast, when an element is referred to as being “directly on,” “directly connected to”, “directly coupled to”, or “immediately adjacent to” another element, there are no intervening elements present.

As utilized herein, the term “substantially” and similar terms are utilized as terms of approximation and not as terms of degree, and are intended to account for the inherent deviations in measured or calculated values that would be recognized by those of ordinary skill in the art. Also, the terms “about,” “approximately,” and similar terms, when utilized herein in connection with a numerical value or a numerical range, are inclusive of the stated value and refer to within an acceptable range of deviation for the particular value as determined by one of ordinary skill in the art, considering the measurement in question and the error associated with measurement of the particular quantity (e.g., the limitations of the measurement system).

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Example embodiments of the present disclosure will now be described with reference to the accompanying drawings. In the drawings, the same or similar reference numerals refer to the same or similar elements throughout. As utilized herein, the utilize of the term “may,” when describing embodiments of the present disclosure, refers to “one or more embodiments of the present disclosure.”

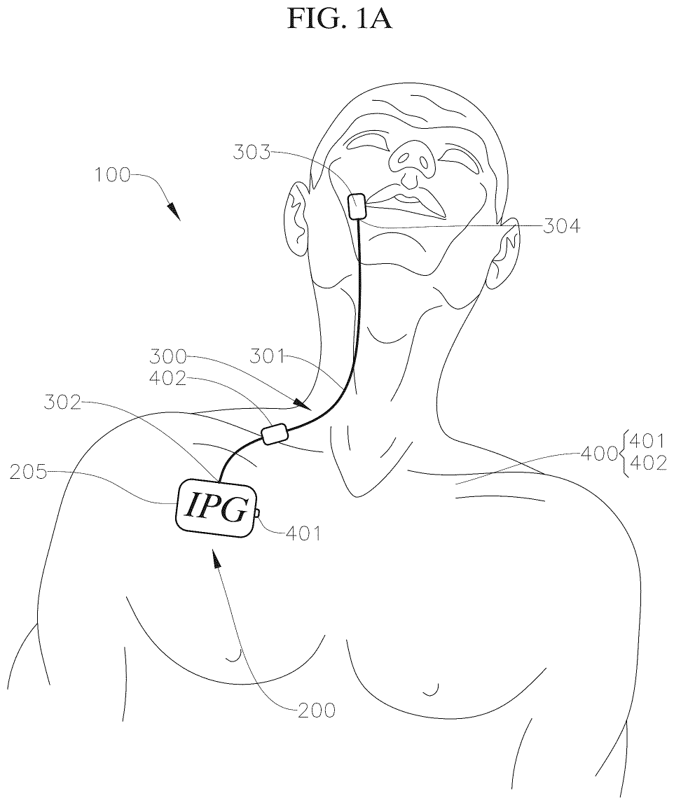

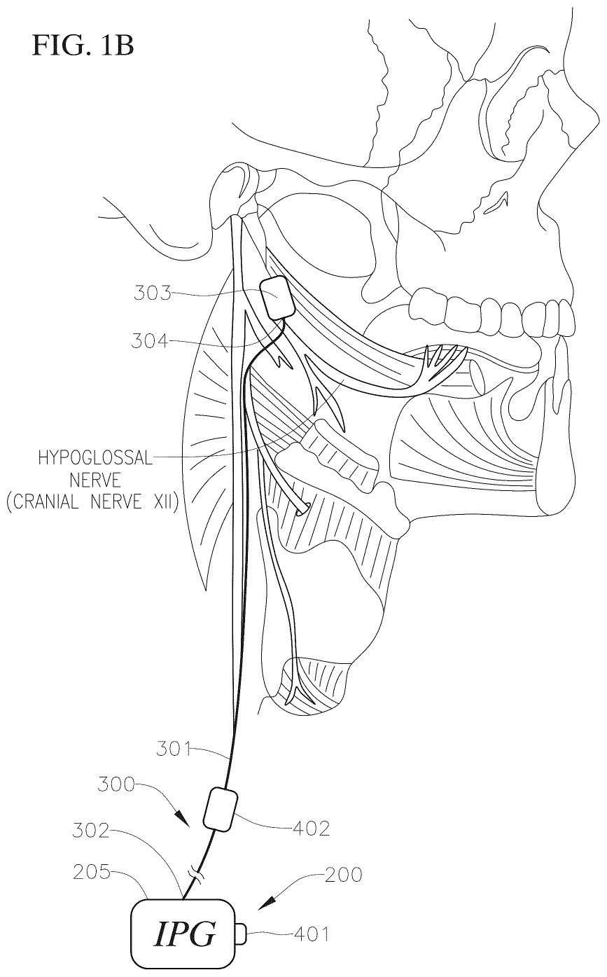

With reference now to FIGS. 1A-1B, a nerve stimulation (NS) device 100 for treating a patient with obstructive sleep apnea according to one embodiment of the present disclosure includes an implantable pulse generator (IPG) 200, a stimulation system 300 coupled to the IPG 200 for stimulating a nerve of the patient that is associated with sleep apnea (e.g., the hypoglossal nerve and/or the ansa cervicalis nerve), and an impedance measurement system 400 coupled to (or in communication with) the IPG 200 for measuring the thoracic impedance (e.g., intra-thoracic impedance or trans-thoracic impedance) of the patient.

In the illustrated embodiment, the stimulation system 300 includes at least one implantable lead 301 having a proximal end 302 coupled to the IPG 200, and at least one electrode (e.g., at least one nerve cuff electrode) 303 coupled to a distal end 304 of the at least one implantable lead 301. When the nerve stimulation (NS) device 100 is implanted in a patient afflicted with obstructive sleep apnea, the IPG 200 may be implanted subcutaneously in the patient's chest (e.g., a subcutaneous pocket over the fascia of the pectoralis major muscle), the at least one implantable lead 301 may be tunneled between the chest wall and the neck over the patient's clavicle, and the electrode 303 contacts (e.g., at least partially surrounds) a nerve associated with sleep apnea (e.g., the hypoglossal nerve (cranial nerve XII) and/or the ansa cervicalis nerve), which is responsible for tongue movement, as described in more detail below. In one or more embodiments, the IPG 200 may be implanted in any other suitable location, such as in the patient's neck or sub-pectorally in the patient. In one or more embodiments, the IPG 200 may be implanted in the patient's neck or chin.

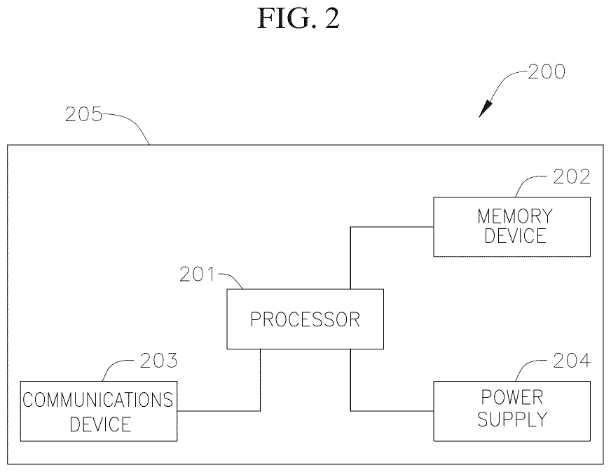

With reference now to FIG. 2, the IPG 200 includes a processor (e.g., a processing circuit) 201, a non-volatile memory device 202 (e.g., flash memory, ferroelectric random-access memory (FeRAM), magnetoresistive random-access memory (MRAM), phase-change memory (PCM), FeFET memory, and/or resistive random-access memory (RRAM)), a communications device 203 (e.g., a receiver and a transmitter, or a transceiver), and a power supply 204 (e.g., a primary battery or an inductively chargeable rechargeable battery). The communications device 203 provides wireless communication links through the skin of the patient to a clinician programmer (CP) device and/or a patient remote (PR) device. Wireless links may include Bluetooth™, Bluetooth Low Energy or other protocols with suitable authentication and encryption to protect patient data. In one or more embodiments, the non-volatile memory device 202, the communications device 203, and the power supply 204 are in communication with each other over the processor 201. Additionally, in the illustrated embodiment, the processor 201, the non-volatile memory device 202, the communications device 203, and the power supply 204 are housed in a housing or a case 205, and the proximal end(s) 302 of the implantable lead(s) 301 extend through opening(s) in the case (e.g., housing) 205 and are connected to dedicated circuitry that provides stimulation pulses as controlled by the processor 201.

The term “processor” is utilized herein to include any combination of hardware, firmware, memory and software, employed to process data or digital signals. The hardware of a processor may include, for example, a microcontroller, application specific integrated circuits (ASICs), general purpose or special purpose central processors (CPUs), digital signal processors (DSPs), graphics processors (GPUs), and programmable logic devices such as field programmable gate arrays (FPGAs). In a processor, as utilized herein, each function is performed either by hardware configured, i.e., hard-wired, to perform that function, or by more general-purpose hardware, such as a CPU, configured to execute instructions stored in a non-transitory storage medium or memory. A processor may contain two or more processors, for example, a processor may include two processors, an FPGA and a CPU, interconnected on a PCB.

With reference again to FIGS. 1A-1B, the impedance measurement system 400 includes at least two electrodes 401, 402 configured to measure the thoracic impedance (e.g., trans-thoracic impedance or intra-thoracic impedance) of the patient. As described in more detail below, the thoracic impedance is a respiratory signal that may be utilized to control the timing of the stimulation delivered by the implantable lead 301 to the nerve associated with sleep apnea (e.g., the hypoglossal nerve and/or the ansa cervicalis nerve). The electrodes 401, 402 may be either implantable electrodes or surface electrodes. For instance, in the illustrated embodiment, the impedance measurement system 400 includes a first electrode 401 on the housing 205 of the IPG 200 and a second electrode 402 on the implantable lead 301. The second electrode 402 may be proximate to the electrode 303 at the distal end 304 of the implantable lead 301 (e.g., the second electrode 402 for measuring thoracic impedance may be proximate to the electrode 303 for delivering a therapeutic stimulation of the nerve associated with sleep apnea). In one or more embodiments, the second electrode 402 may be at an intermediate position along the implantable lead 301 between the case 205 and the electrode 303 at the distal end 304 of the implantable lead 301. In one or more embodiments, the second electrode 402 may be at the proximal end 302 of the implantable lead 301. In one or more embodiments, the second electrode 402 may be coupled to the IPG 200 by an implantable lead separate from the implantable lead 301 on which the electrode 303 for delivering a therapeutic stimulation of the nerve is located. In one or more embodiments, the electrode 303 for delivering stimulation to the nerve associated with sleep apnea may also be utilized to measure the thoracic impedance (e.g., one electrode may be both a stimulation electrode and a thoracic impedance measurement electrode). In one or more embodiments, both the first electrode 401 and the second electrode 402 may be coupled to the IPG 200 by implantable leads separate from the implantable lead 301 on which the electrode 303 for delivering a therapeutic stimulation of the nerve is located. In one or more embodiments, both the first electrode 401 and the second electrode 402 may be surface electrodes (e.g., electrode patches) configured to be adhered or otherwise attached to the exterior of the chest or abdomen of the patient. In one or more embodiments in which the electrodes are surface electrodes, the electrodes may be configured to wireless transmit (directly or indirectly) the thoracic impedance measurement to the IPG 200. In one or more embodiments, the impedance measurement system 400 may include four electrodes that may be implantable electrodes, surface electrodes, or a combination of implantable electrodes and surface electrodes.

In one or more embodiments, the electrodes 401, 402 may not be physically connected to the IPG 200 (e.g., the electrodes 401, 402 for measuring the thoracic impedance of the patient may be remote from the IPG 200). In one or more embodiments in which the electrodes 401, 402 are remote from the IPG 200, the electrodes 401, 402 may be configured to wirelessly transmit, either directly or indirectly, sensor information (e.g., a signal representing the thoracic impedance of the patient) to the IPG 200.

The non-volatile memory device 202 of the IPG 200 includes computer-readable instructions which, when executed by the processor 201, cause the IPG 200 to deliver stimulation to the electrode 303 based on the thoracic impedance measured or determined by the electrodes 401, 402. For example, in one or more embodiments, the computer-readable instructions, when executed by the processor 201, cause the IPG 200 to determine when the patient begins inhaling and stops inhaling (or begins exhaling) based on the values of the thoracic impedance. In general, the thoracic impedance changes (in both magnitude and direction) as the volume of the patient's lung changes and thus the thoracic impedance may be utilized to determine the patient's respiratory cycle (i.e., the thoracic impedance increases with increasing lung volume due to the presence of additional air in the lungs, and the thoracic impedance decreases with decreasing lung volume due the presence of less air in the lungs). Accordingly, in one or more embodiments, the computer-readable instructions, when executed by the processor 201, cause the IPG 200 to deliver stimulation to the electrode 303 (and the nerve associated with sleep apnea (e.g., the hypoglossal nerve and/or the ansa cervicalis nerve) in response to the value of the thoracic impedance indicating that the patient has started inhaling or is about to start inhaling (e.g., in response to the value of the thoracic impedance increasing from a minimum value), and cause the IPG 200 to cease delivering stimulation to the electrode 303 (and the nerve associated with sleep apnea) in response to the value of the thoracic impedance indicating that the patient has stopped inhaling or started exhaling (e.g., in response to the value of the thoracic impedance reaching a maximum value or starting to decrease from a maximum value). The hypoglossal nerve innervates all the extrinsic and intrinsic muscles of the tongue except for the palatoglossus, which is innervated by the vagus nerve. Accordingly, stimulating the hypoglossal nerve causes the patient's tongue to move forward, which relieves upper airway obstruction and thereby permits the patient to breath freely. Stimulating the ansa cervicalis nerve increases pharyngeal patency and pulls the pharyngeal structures downward. In this manner, the computer-readable instructions are configured to utilize the value of the thoracic impedance to control the timing of the delivery of the stimulation to the electrode 303 and the nerve associated with sleep apnea (e.g., the computer-readable instructions, when executed by the processor 201, cause the IPG 200 to start and stop the delivery of the stimulation to the nerve based on the values of the thoracic impedance).

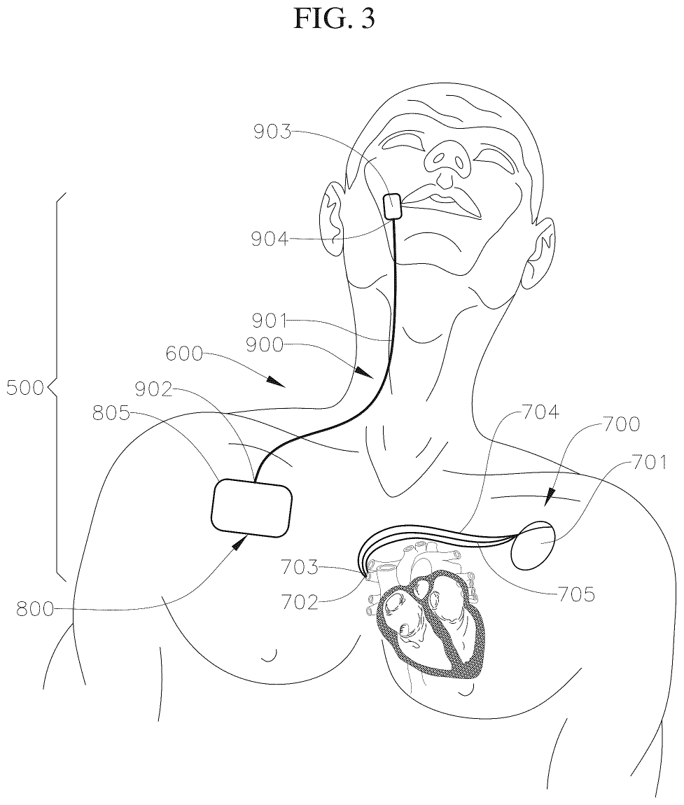

With reference now to FIG. 3, a system 500 for treating a patient with obstructive sleep apnea according to another embodiment of the present disclosure includes a nerve stimulation (NS) device 600 and a cardiac resynchronization therapy defibrillator (CRT-D) or an implantable cardioverter defibrillator (ICD) 700 in communication with the NS device 600. The CRT-D and the ICD 700 are implantable devices configured to detect and stop irregular heartbeats (i.e., arrhythmias) or excessively fast heartbeats (e.g., ventricular tachycardia or ventricular fibrillation) in the patient. The CRT-D and the ICD 700 each include a generator 701 and electrodes 702, 703 connected to the generator 701 with leads 704, 705, respectively. The leads 704, 705 extend into the right atrium and ventricle of the patient's heart.

In the illustrated embodiment, the NS device 600 includes an implantable pulse generator (IPG) 800 and a stimulation system 900 coupled to the IPG 800 for stimulating a nerve of the patient associated with sleep apnea (e.g., the hypoglossal nerve and/or the ansa cervicalis nerve). Unlike the embodiment of the NS device 100 depicted in FIGS. 1A-1B, the NS device 600 may not include an impedance measurement system for measuring the thoracic impedance (e.g., intra-thoracic impedance or trans-thoracic impedance) of the patient. In one or more embodiments, the CRT-D or the ICD 700 may measure or determine the thoracic impedance (e.g., the trans-thoracic impedance or the intra-thoracic impedance) of the patient, and the NS device 600 may receive the thoracic impedance (directly or indirectly) from the CRT-D or the ICD 700.

In the illustrated embodiment, the stimulation system 900 includes at least one implantable lead 901 having a proximal end 902 coupled to the IPG 800, and at least one electrode (e.g., at least one nerve cuff electrode) 903 coupled to a distal end 904 of the at least one implantable lead 901. When the NS device 600 is implanted in a patient afflicted with obstructive sleep apnea, the IPG 800 may be implanted subcutaneously in the patient's chest (e.g., a subcutaneous pocket over the fascia of the pectoralis major muscle), the at least one implantable lead 901 may be tunneled between the chest wall and the neck over the patient's clavicle, and the electrode 903 contacts (e.g., at least partially surrounds) a nerve associated with sleep apnea (e.g., the hypoglossal nerve (cranial nerve XII) and/or the ansa cervicalis nerve). The hypoglossal nerve is responsible for tongue movement, as described in more detail below. In one or more embodiments, the IPG 800 may be implanted in any other suitable location, such as in the patient's neck or sub-pectorally in the patient.

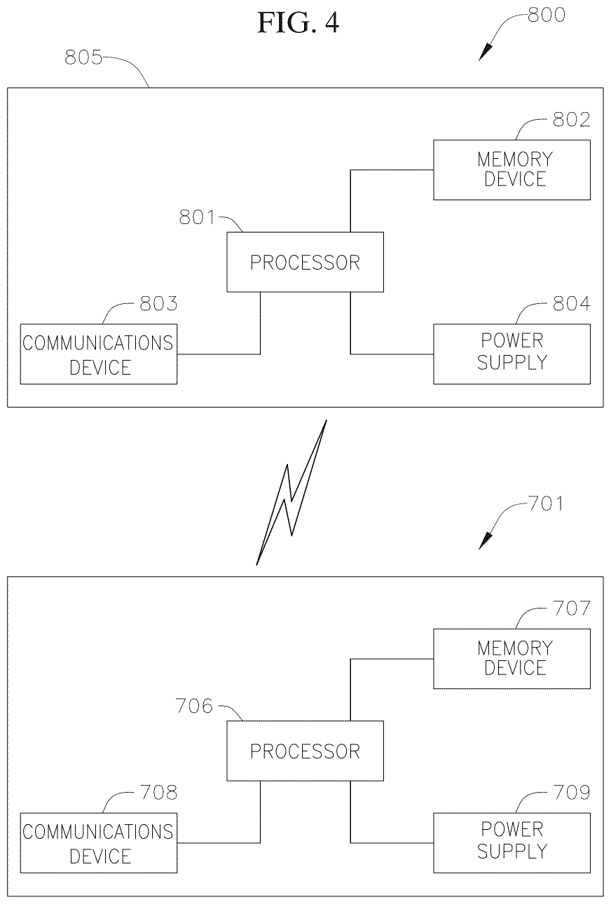

With reference now to FIG. 4, the generator 701 of the CRT-D or the ICD 700 includes a processor (e.g., a processing circuit) 706, a non-volatile memory device 707 (e.g., flash memory, ferroelectric random-access memory (FeRAM), magnetoresistive random-access memory (MRAM), phase-change memory (PCM), FeFET memory, and/or resistive random-access memory (RRAM)), a communications device 708 (e.g., a receiver and a transmitter, or a transceiver), and a power supply 709 (e.g., a primary battery or an inductively chargeable rechargeable battery). The communications device 708 provides wireless communication links to the IPG 800. Wireless links may include Bluetooth™, Bluetooth Low Energy or other protocols with suitable authentication and encryption to protect patient data. In one or more embodiments, the non-volatile memory device 707, the communications device 708, and the power supply 709 are in communication with each other over the processor 706.

In one or more embodiments, the non-volatile memory device 707 of the CRT-D or the ICD 700 includes computer-readable instructions which, when executed by the processor 706, cause the CRT-D or the ICD 700 to calculate or determine the thoracic impedance of the patient utilizing the electrodes 702, 703. Additionally, in one or more embodiments, the non-volatile memory device 707 of the CRT-D or the ICD 700 includes computer-readable instructions which, when executed by the processor 706, cause the CRT-D or the ICD 700 to transmit the thoracic impedance to the IPG 800 utilizing the communications device 708.

With continued reference to FIG. 4, the IPG 800 includes a processor (e.g., a processing circuit) 801, a non-volatile memory device 802 (e.g., flash memory, ferroelectric random-access memory (FeRAM), magnetoresistive random-access memory (MRAM), phase-change memory (PCM), FeFET memory, and/or resistive random-access memory (RRAM)), a communications device 803 (e.g., a receiver and a transmitter, or a transceiver), and a power supply 804 (e.g., a primary battery or an inductively chargeable rechargeable battery). The communications device 803 provides wireless communication links through the skin of the patient to a clinician programmer (CP) device and/or a patient remote (PR) device. Wireless links may include Bluetooth™, Bluetooth Low Energy or other protocols with suitable authentication and encryption to protect patient data. In one or more embodiments, the communications device 803 may provide a wireless communication link with the CRT device or the ICD device 700 implanted in the patient. In one or more embodiments, the non-volatile memory device 802, the communications device 803, and the power supply 804 are in communication with each other over the processor 801. Additionally, in the illustrated embodiment, the processor 801, the non-volatile memory device 802, the communications device 803, and the power supply 804 are housed in a housing or a case 805, and the proximal end(s) 902 of the implantable lead(s) 901 extend through opening(s) in the case (e.g., housing) 805 and are connected to dedicated circuitry that provides stimulation pulses as controlled by the processor 801.

The non-volatile memory device 802 of the IPG 800 includes computer-readable instructions which, when executed by the processor 801, cause the IPG 800 to deliver stimulation to the electrode 903 based on the thoracic impedance measured or determined by the CRT-D or the ICD 700. For example, in one or more embodiments, the computer-readable instructions, when executed by the processor 801, cause the IPG 800 to determine when the patient begins inhaling and stops inhaling (or begins exhaling) based on the values of the thoracic impedance. As described above, the thoracic impedance changes (in both magnitude and direction) as the volume of the patient's lung changes and thus the thoracic impedance may be utilized to determine the patient's respiratory cycle (i.e., the thoracic impedance increases with increasing lung volume due to the presence of additional air in the lungs, and the thoracic impedance decreases with decreasing lung volume due the presence of less air in the lungs). Accordingly, in one or more embodiments, the computer-readable instructions cause the IPG 800 to deliver stimulation to the electrode 903 (and a nerve associated with sleep apnea (e.g., the hypoglossal nerve and/or the ansa cervicalis nerve) in response to the value of the thoracic impedance indicating that the patient has started inhaling (e.g., in response to the value of the thoracic impedance increasing from a minimum value), and cause the IPG 800 to cease delivering stimulation to the electrode 903 (and the nerve associated with sleep apnea) in response to the value of the thoracic impedance indicating that the patient has stopped inhaling or started exhaling (e.g., in response to the value of the thoracic impedance reaching a maximum value or starting to decrease from a maximum value). As described above, stimulating the hypoglossal nerve causes the patient's tongue to move forward, which relieves upper airway obstruction and thereby permits the patient to breath freely. Additionally, as described above, stimulating the ansa cervicalis nerve pulls the pharyngeal structures downward. In this manner, the computer-readable instructions are configured to utilize the value of the thoracic impedance to control the timing of the delivery of the stimulation to the electrode 903 and the nerve associated with sleep apnea (e.g., the computer-readable instructions, when executed by the processor, cause the IPG 800 to start and stop the delivery of the stimulation to the nerve based on the values of the thoracic impedance).

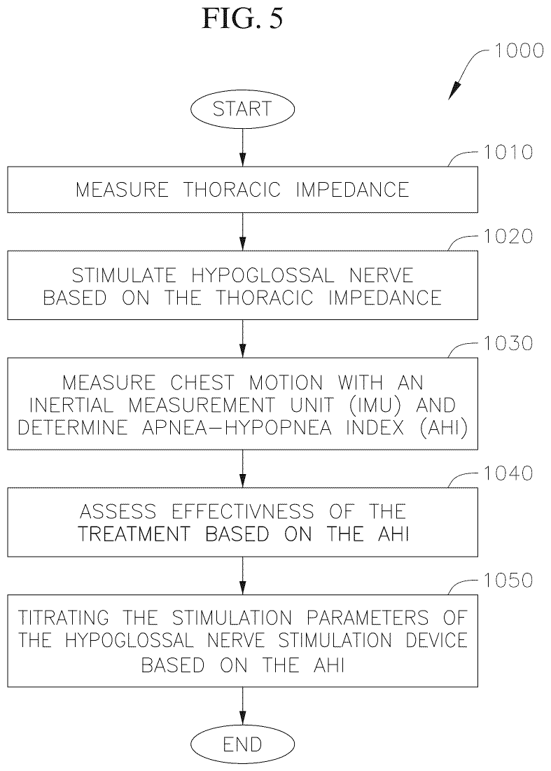

FIG. 5 is a flowchart illustrating tasks of a method 1000 of treating a patient afflicted by obstructive sleep apnea. In the illustrated embodiment, the method 1000 includes a task 1010 of measuring or otherwise determining the thoracic impedance of the patient (i.e., the electrical impedance of the patient's thorax, which is the region between the abdomen inferiorly and the root of the neck superiorly). The task 1010 of measuring the impedance of the patient's thorax may be performed with two or more electrodes. In one or more embodiments, the two or more electrodes may be surface electrodes adhered to the patient's chest and/or abdomen. In one or more embodiments, the two or more electrodes may be implantable electrodes that are implanted in the patient. Depending on the location of the electrodes, the task 1010 may include measuring or determining the thoracic impedance, the intra-thoracic impedance, or the trans-thoracic impedance of the patient. In one or more embodiments, the two or more electrodes may include a first electrode on a housing of a nerve stimulation device and a second electrode connected to the housing via an electrical lead. In one or more embodiments, the two or more electrodes may include a first electrode connected to a housing of a nerve stimulation device via a first electrical lead, and a second electrode connected to the housing via a second electrical lead. In one or more embodiments, the task 1010 may be performed by a cardiac resynchronization therapy defibrillator (CRT-D) or an implantable cardiovert defibrillator (ICD) in the patient. The thoracic impedance changes (in both magnitude and direction) as the volume of the patient's lung changes (i.e., the thoracic impedance increases with increasing lung volume due to the presence of additional air in the lungs, and the thoracic impedance decreases with decreasing lung volume due the presence of less air in the lungs). Accordingly, the thoracic impedance may be utilized to determine the patient's respiratory cycle. The thoracic impedance may also be utilized to detect congestive heart failure, which is a comorbidity associated with obstructive sleep apnea.

In the illustrated embodiment, the method 1000 also includes a task 1020 of stimulating one or more nerves of the patient that are associated with sleep apnea (e.g., the hypoglossal nerve and/or the ansa cervicalis nerve) with a nerve stimulation device based on the thoracic impedance of the patient (determined in task 1010) that indicates that the patient is inhaling. That is, the task 1020 includes utilizing the thoracic impedance to determine when the patient is inhaling and stimulating the nerve of the patient that is associated with sleep apnea when the patient is inhaling. The hypoglossal nerve innervates all the extrinsic and intrinsic muscles of the tongue except for the palatoglossus, which is innervated by the vagus nerve. Accordingly, stimulating the hypoglossal nerve causes the patient's tongue to move forward, which relieves upper airway obstruction and thereby permits the patient to breath freely. The stimulation of the hypoglossal nerve may be performed with a hypoglossal nerve stimulation device. Stimulating the ansa cervicalis nerve pulls the pharyngeal structures of the patient downward, which may relieve the patient of symptoms associated with sleep apnea.

In the illustrated embodiment, the method 1000 also includes a task 1030 of measuring the chest motion of the patient with an inertial measurement unit (IMU). The task 1030 may also include utilizing the chest motion of the patient to measure (or estimate) the respiratory phase of the patient and to measure (or estimate) the respiratory flow and the respiratory effort of the patient. In one or more embodiments, the task 1030 may also include utilizing these factors (respiratory flow and respiratory effort) to detect apneic and hypopneic respiratory events in real-time and to calculate the apnea-hypopnea index (AHI), which is the average number of apneas and hypopneas that occur per hour of sleep.

In the illustrated embodiment, the method 1000 also includes a task 1040 of assessing the effectiveness of the treatment on the patient's obstructive sleep apnea based on the AHI determined in task 1030 (i.e., the lower the AHI value or the more the AHI value has been reduced, the greater the effectiveness of the treatment).

In the illustrated embodiment, the method 1000 also includes a task 1050 of titrating the stimulation parameters of the nerve stimulation device based on the AHI determined in task 1030 to further improve the therapeutic effectiveness (e.g., adjusting the parameters for stimulating the patient's nerve based on the AHI). In one or more embodiments, the task 1050 of titrating the stimulation parameters based on the AHI may be performed under the guidance of a clinician or performed automatically via machine learning and/or artificial intelligence algorithms.

In one or more embodiments, the method 1000 may not include the task 1030 of measuring the chest motion with the IMU and calculating the AHI, the task 1040 of assessing the effectiveness of the treatment on the patient's obstructive sleep apnea based on the AHI, or the task 1050 titrating the stimulation parameters based on the AHI.

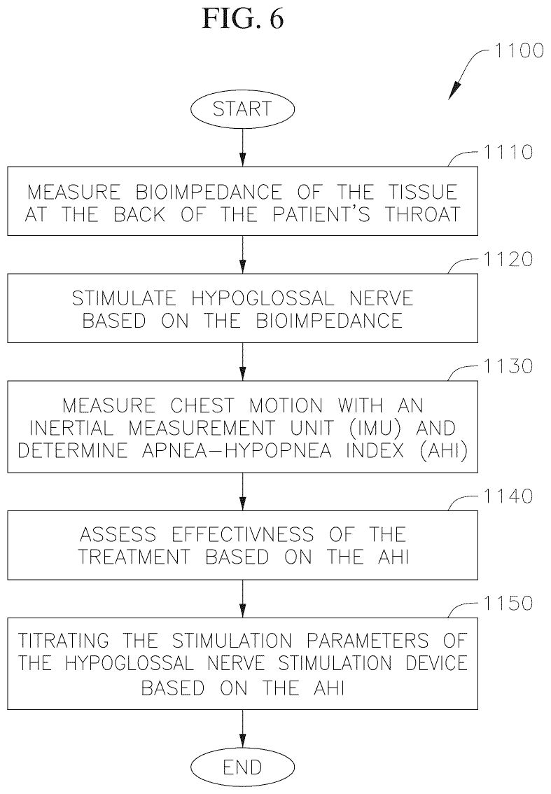

FIG. 6 is a flowchart illustrating tasks of a method 1100 of treating a patient afflicted by obstructive sleep apnea according to another embodiment of the present disclosure. In the illustrated embodiment, the method includes a task 1110 of measuring or otherwise determining the bioimpedance of the tissue at the back of the patient's throat. The task 1110 of measuring the bioimpedance of the tissue at the back of the patient's throat may be performed with one or more bioimpedance electrodes positioned in the tissue near the region of the airway that typically closes during sleep apnea. The bioimpedance changes as the patient's airway opens and closes (i.e., the bioimpedance increases as the airway opens and the bioimpedance decreases as the airway closes). Accordingly, the bioimpedance may be utilized to determine the extent of opening or closure of the patient's airway.

In the illustrated embodiment, the method 1100 also includes a task 1120 of stimulating a nerve of the patient associated with sleep apnea (e.g., the hypoglossal nerve and/or the ansa cervicalis) with a nerve stimulation device based on the bioimpedance of the patient (determined in task 1110) that indicates that the patient is inhaling. That is, the task 1120 includes utilizing the bioimpedance to determine when the patient is inhaling and stimulating the nerve of the patient when the patient is inhaling. Stimulating the hypoglossal nerve causes the patient's tongue to move, which relieves upper airway obstruction and thereby permits the patient to breath freely. Stimulating the ansa cervicalis nerve pulls the pharyngeal structures of the patient downward, which may relieve the patient of symptoms associated with sleep apnea.

In the illustrated embodiment, the method 1100 also includes a task 1130 of measuring the chest motion of the patient with an inertial measurement unit (IMU). The task 1130 may also include utilizing the chest motion of the patient to measure (or estimate) the respiratory phase of the patient and to measure (or estimate) the respiratory flow and the respiratory effort of the patient. In one or more embodiments, the task 1130 may also include utilizing these factors (respiratory flow and respiratory effort) to detect apneic and hyopneic respiratory events in real-time and to calculate the apnea-hypopnea index (AHI), which is the average number of apneas and hypopneas that occur per hour of sleep.

In the illustrated embodiment, the method 1100 also includes a task 1140 of assessing the effectiveness of the treatment on the patient's obstructive sleep apnea based on the AHI determined in task 1130 (i.e., the lower the AHI value or the more the AHI value has been reduced, the greater the effectiveness of the treatment).

In the illustrated embodiment, the method 1100 also includes a task 1150 of titrating the stimulation parameters of the nerve stimulation device based on the AHI determined in task 1130 to further improve the therapeutic effectiveness (e.g., adjusting the parameters for stimulating the patient's nerve associated with sleep apnea based on the AHI). In one or more embodiments, the task 1150 of titrating the stimulation parameters based on the AHI may be performed under the guidance of a clinician or performed automatically via machine learning and/or artificial intelligence algorithms.

In one or more embodiments, the method 1100 may not include the task 1130 of measuring the chest motion with the IMU and calculating the AHI, the task 1140 of assessing the effectiveness of the treatment on the patient's obstructive sleep apnea based on the AHI, or the task 1150 titrating the stimulation parameters based on the AHI.

The system and/or any other relevant devices or components according to embodiments of the present disclosure described herein may be implemented utilizing any suitable hardware, firmware (e.g., an application-specific integrated circuit), software, or a combination of software, firmware, and hardware. For example, the one or more suitable components of the system may be formed on one integrated circuit (IC) chip or on separate IC chips. Further, the one or more suitable components of the system may be implemented on a flexible printed circuit film, a tape carrier package (TCP), a printed circuit board (PCB), or formed on one substrate. Further, the one or more suitable components of the system may be a process or thread, running on one or more processors, in one or more computing devices, executing computer program instructions and interacting with other system components for performing the one or more suitable functionalities described herein. The computer program instructions are stored in a memory which may be implemented in a computing device utilizing a standard memory device, such as, for example, a random access memory (RAM). The computer program instructions may also be stored in other non-transitory computer readable media such as, for example, a flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although some embodiments of the present disclosure have been disclosed herein, the present disclosure is not limited thereto, and the scope of the present disclosure is defined by the appended claims and equivalents thereof.

What is claimed is:

1. A method of treating obstructive sleep apnea in a patient, the method comprising:
measuring, with a plurality of electrodes, thoracic impedance of the patient; and
stimulating, with a nerve stimulation device implanted in the patient, a nerve of the patient based on the thoracic impedance, wherein the nerve is associated with sleep apnea.

2. The method of claim 1, wherein the nerve is a hypoglossal nerve of the patient.

3. The method of claim 1, wherein the nerve is an ansa cervicalis nerve of the patient.

4. The method of claim 1, wherein the stimulating the nerve is performed in response to a value of the thoracic impedance indicating the patient started to inhale or is about to inhale.

5. The method of claim 1, further comprising wirelessly transmitting the thoracic impedance from the plurality of electrodes to the nerve stimulation device.

6. The method of claim 5, wherein the plurality of electrodes is attached to the patient's chest.

7. The method of claim 5, wherein the plurality of electrodes is attached to the patient's abdomen.

8. The method of claim 1, wherein the plurality of electrodes is implanted in the patient.

9. The method of claim 1, wherein the plurality of electrodes comprises a first electrode on a housing of an implantable pulse generator of the nerve stimulation device and a second electrode coupled to the nerve stimulation device and connected to the housing by a first lead.

10. The method of claim 9, wherein the stimulating is performed with the second electrode.

11. The method of claim 9, wherein the stimulating is performed with a third electrode coupled to the housing of the nerve stimulation device by a second lead separate from the first lead.

12. The method of claim 9, wherein the stimulating is performed with a third electrode coupled to the housing of the nerve stimulation device by the first lead.

13. The method of claim 1, wherein the measuring comprises measuring trans-thoracic impedance of the patient.

14. The method of claim 1, wherein the measuring comprises measuring intra-thoracic impedance of the patient.

15. The method of claim 1, further comprising measuring, with an inertial measurement unit of the nerve stimulation device, movement of the patient's chest.

16. The method of claim 15, wherein the stimulating is further based on the movement of the patient's chest.

17. The method of claim 1, further comprising measuring, with an inertial measurement unit, motion of the patient's chest.

18. The method of claim 17, further comprising titrating stimulation parameters of the nerve stimulation device based on the motion of the patient's chest.

19. A method of treating obstructive sleep apnea in a patient, the method comprising:
measuring, with at least one bioimpedance electrode, bioimpedance of tissue at a back of the patient's throat;
stimulating, with a nerve stimulation device implanted in the patient, a nerve of patient based on the bioimpedance, wherein the nerve is associated with sleep apnea.

20. The method of claim 19, wherein the nerve is a hypoglossal nerve of the patient.

21. The method of claim 19, wherein the nerve is an ansa cervicalis nerve of the patient.

22. The method of claim 19, wherein the stimulating the nerve is performed in response to a value of the bioimpedance indicating the patient started to inhale or is about to inhale.

23. A nerve stimulation device configured to treat obstructive sleep apnea in a patient, the nerve stimulation device comprising:
a housing;
a processor in the housing;
a non-volatile memory device in the housing;
a controller in the housing;
a power supply in the housing; and
a plurality of electrodes coupled to the housing,
wherein the non-volatile memory device comprises computer-readable instructions which, when executed by the controller, cause the plurality of electrodes to measure thoracic impedance of the patient and to deliver electrical stimulation to a nerve of the patient based on the thoracic impedance, wherein the nerve is associated with sleep apnea.

24. The nerve stimulation device of claim 23, wherein the nerve stimulation device is a hypoglossal nerve stimulation device.

25. The nerve stimulation device of claim 23, wherein the computer-readable instructions, when executed by the controller, cause the plurality of electrodes to deliver the electrical stimulation to the nerve in response to a value of the thoracic impedance indicating the patient started to inhale or is about to inhale.

26. The nerve stimulation device of claim 23, wherein the computer-readable instructions, when executed by the controller, cause the controller to wirelessly receive the thoracic impedance from the plurality of electrodes.

27. The nerve stimulation device of claim 26, wherein the plurality of electrodes comprises a plurality of surface electrodes configured to be attached to the patient's chest or abdomen.

28. The nerve stimulation device of claim 23, wherein the plurality of electrodes comprises a plurality of implantable electrodes.

29. The nerve stimulation device of claim 23, wherein the plurality of electrodes comprises a first electrode on the housing and a second electrode coupled to the housing by a lead.

30. The nerve stimulation device of claim 23, wherein the plurality of electrodes comprises at least one first electrode coupled to the housing by a first lead and at least one second electrode coupled to the housing by a second lead.

31. The nerve stimulation device of claim 23, wherein the plurality of electrodes comprises two electrodes.

32. The nerve stimulation device of claim 23, wherein the plurality of electrodes comprises at least three electrodes.
